(19) United States
(12) Patent Application Publication
(10) Pub. No.: US 2007/0142900 A1
(43) Pub. Date: Jun. 21, 2007

(54) STENT INCLUDING A PORTAL AND METHODS OF USE THEREOF

(76) Inventor: Malur R. Balaji, Pittsford, NY (US)

Correspondence Address: BURNS & LEVINSON, LLP, 125 SUMMER STREET, BOSTON, MA, 02110, US

(21) Appl. No.: 11/566,410

(22) Filed: Dec. 4, 2006

Related U.S. Application Data
(60) Provisional application No. 60/742,316, filed on Dec. 5, 2005.
(60) Provisional application No. 60/742,802, filed on Dec. 5, 2005.

(51) Int. Cl. A61F 2/90; A61F 2/84

(52) U.S. Cl. 623/1.16; 623/1.34

(57) ABSTRACT

Apparatus and methods for endovascular procedures for the placement of a stent in a bifurcated artery.

### CROSS REFERENCE TO RELATED APPLICATIONS

This application claims priority of U.S. Provisional Application 60/742,802 entitled STENT INCLUDING A PORTAL AND METHODS OF USE THEREOF, filed on Dec. 5, 2005, and of U.S. Provisional Application 60/742,316, entitled STENTS WITH BEVELED ENDS AND METHODS OF THE USE THEREOF, also filed on Dec. 5, 2005, both of which are hereby incorporated by reference herein.

### BACKGROUND

These teachings relates generally to apparatus and methods for endovascular procedures for the placement of a stent in a bifurcated artery.

Stents are generally tubular-shaped devices which function to hold open a segment of a blood vessel or other anatomical lumen. They are particularly suitable for use to support and hold back a dissected arterial lining which can occlude the fluid passageway therethrough.

Stenting is the permanent placement of a small, latticed tube inside an anatomical lumen to provide structural support and to keep the lumen (hollow channel) open to maintain blood flow. The stenting procedure involves passing a collapsed stent into the artery to the site that requires support. The lattices of the stent are then allowed to expand, increasing the diameter of the stent. The expanded stent is then left permanently in place in the vessel.

Various means have been described to deliver and implant stents. One method frequently described for delivering a stent to a desired intraluminal location includes mounting the expandable stent on an expandable member, such as a balloon, provided on the distal end of an intravascular catheter, advancing the catheter to the desired location within the patient's body lumen, inflating the balloon on the catheter to expand the stent into a permanent expanded condition and then deflating the balloon and removing the catheter.

The current design of vascular stents is a tube whose wall is constructed of an expandable, structural, open-lattice made of a material such as nickel titanium (NiTinol), stainless steel, or other materials.

When deployed in a bifurcated artery, the axis of the stent is aligned with the trunk and one of the branches of the artery. Flow to the other branch is accomplished by allowing the blood to pass through the interstices (openings) of the structural lattice of the stent, as shown in FIG. 1. Thrombosis (clotting) can occur when the lattices interfere with the blood flow, such as when particles in the blood become attached to the lattices of the stent. This clotting can impede the flow of blood to the branch.

In addition, the presence of the stent wall at the mouth of the branch can create an obstacle to future endovascular procedures involving the branch. For example, to perform a balloon angioplasty in the branch, the angioplasty catheter must first breach the wall of the stent.

It is therefore a need to provide a stent that allows unimpeded flow of blood to a branch artery.

It is a further need to provide a stent that allows performing future endovascular procedures involving the branch artery.

### BRIEF SUMMARY

In one embodiment, the stent of these teachings includes a substantially cylindrical structure comprising a number of interconnected elements (a lattice) where an outer surface of the substantially cylindrical structure has an orifice (portal) capable of allowing flow between an interior volume of the substantially cylindrical structure and an external volume. In one embodiment, the orifice is dimensioned to allow cannulation (insertion of a catherer, in one instance) of a branch vessel where a proximal end of the branch vessel is located substantially at a location of the orifice. In one embodiment, the size of the portal is slightly smaller than the diameter of the branch vessel (artery) to allow the stent wall to contain plaque at the proximal end (opening of) the branch vessel. In one embodiment, an area of the outer surface (wall) of the stent, where the area is circumferential to the orifice, includes one or more pairs of markers located a direction substantially parallel to a central axis of the substantially cylindrical surface, and at least another pair of markers located in a direction substantially perpendicular to the central axis. The markers include material that is detectable by the imaging method used to guide the placement of the stent. For example, in one instance, the stent is constructed using Nitinol and the markers are constructed using tantalum.

One embodiment of the stent of these teachings allows blood flow through the length of the stent and also provides an open portal (orifice) in the wall of the stent that accommodates flow to a branch artery. The orifice prevents thrombosis by allowing the high resistance flow to be perfused to the branch without passing through the latticed wall of the stent.

In one embodiment, the method of these teachings includes the deployment of the stent of these teachings in a bifurcated artery such that the long axis of the stent is aligned with the trunk and one branch of the artery and the portal is aligned with the other branch artery

For a better understanding of the present teachings, together with other and further needs thereof, reference is made to the accompanying drawings and detailed description and its scope will be pointed out in the appended claims.

### BRIEF DESCRIPTION OF THE DRAWINGS

FIG. 1 shows a cross sectional view of a bifurcated artery with a conventional stent;

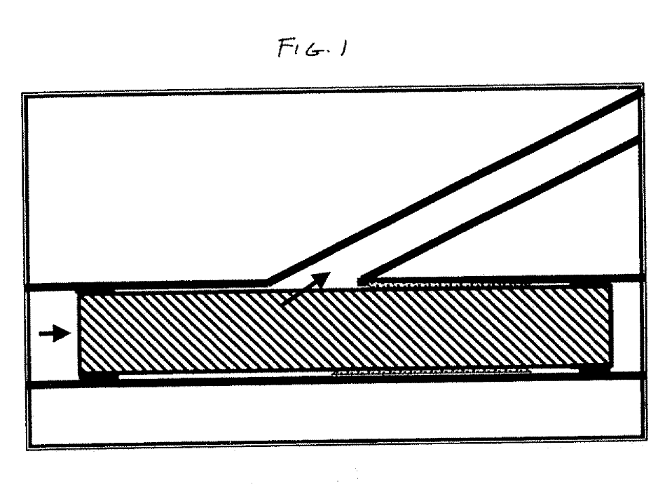

FIG. 2 is shows a cross sectional view of a bifurcated artery with a stent of these teachings;

FIG. 3 shows another embodiment of the stent of these teachings holding open an artery; and

FIG. 4 depicts an application of an embodiment of the stent of these teachings.

### DETAILED DESCRIPTION

In one embodiment, the stent of these teachings includes a substantially cylindrical structure comprising a number of interconnected elements (a lattice) where an outer surface of the substantially cylindrical structure has an orifice (portal) capable of allowing flow between an interior volume of the substantially cylindrical structure and an external volume. In one embodiment, the orifice is dimensioned to allow cannulation (insertion of a catherer, in one instance) of a branch vessel where a proximal end of the branch vessel is located substantially at a location of the orifice. In one embodiment, the size of the portal is slightly smaller than the diameter of the branch vessel (artery) to allow the stent wall to contain plaque at the proximal end (opening of) the branch vessel. The orifice is dimensioned so that sufficient flow is provided to the branch vessel. In one embodiment, an area of the outer surface (wall) of the stent, where the area is circumferential to the orifice, includes one or more pairs of markers located a direction substantially parallel to a central axis of the substantially cylindrical surface, and at least another pair of markers located in a direction substantially perpendicular to the central axis. The markers include material that is detectable by the imaging method used to guide the placement of the stent. For example, in one instance, the stent is constructed using Nitinol and the markers are constructed using tantalum.

The stent of these teachings includes a structural lattice (a number of interconnected elements) such as, but not limited to, the lattice disclosed in U.S. Pat. No. 6,432,133 or in U.S. Pat. No. 5,354,308, both of which are incorporated by reference herein. The dimensions of the stent of these teachings are chosen to accommodate placement within vascular vessels.

The stent of these teachings may be constructed of any material that permits the structure to be expandable, rigid upon expansion, and that is compatible with the use as a stent in a vessel of the human body.

In one embodiment, the stent of these teachings is fabricated of a shape memory alloy, which is encapsulated in its final diametric dimension, and the encapsulated intraluminal stent-graft is manipulated into its reduced diametric dimension and radially expanded in vivo under the influence of a transformation.

The term “shape memory” is used in the art to describe the property of a material to recover a pre-programmed shape after deformation of a shape memory alloy in its martensitic phase and exposing the alloy to a temperature excursion through its austenite transformation temperature, at which temperature the alloy begins to revert to the austenite phase and recover its preprogrammed shape. The term “pseudoelasticity” is used to describe a property of shape memory alloys where the alloy is stressed at a temperature above the transformation temperature of the alloy and stress-induced martensite is formed above the normal martensite formation temperature. Because it has been formed above its normal temperature, stress-induced martensite reverts immediately to undeformed austenite as soon as the stress is removed, provided the temperature remains above the transformation temperature.

Shape memory alloys are a group of metallic materials that demonstrate the ability to return to a defined shape or size when subjected to certain thermal or stress conditions. Shape memory alloys are generally capable of being plastically deformed at a relatively low temperature and, upon exposure to a relatively higher temperature, return to the defined shape or size prior to the deformation. Shape memory alloys may be further defined as one that yields a thermoplastic martensite. A shape memory alloy which yields a thermoplastic martensite undergoes a martensitic transformation of a type that permits the alloy to be deformed by a twinning mechanism below the martensitic transformation temperature. The deformation is then reversed when the twinned structure reverts upon heating to the parent austenite phase. The austenite phase occurs when the material is at a low strain state and occurs at a given temperature. The martensite phase may be either temperature-induced martensite (TIM) or stress-induced martensite (SIM).

In one embodiment, the stent of these teachings utilizes a binary, equiatomic nickel-titanium alloy because of its biocompatibility and because such an alloy exhibits a transformation temperature within the range of physiologically-compatible temperatures.

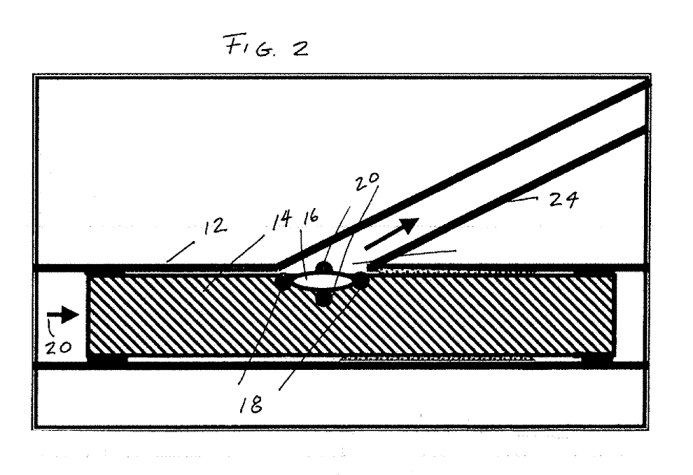

FIG. 2 shows a cross sectional view of a bifurcated artery 12 in which a stent 14 of these teachings has been inserted. The stent of these teachings has a portal 16, which in this embodiment is substantially elliptical. The portal 16 has two markers 18 that are located in a direction substantially parallel to a central axis 20 of the stent 14 and two other markers 22 that are substantially perpendicular to the central axis 20 of the stent 14. The portal 16 is substantially located at the proximal end of the branch artery 24. In the embodiment shown, the longitudinal markers 18 are provided along the long axis of the portal and the transverse markers 22 are provided along the short axis of the portal.

The markers 18, 22 can be detected by the imaging technique used to guide the delivering of the stent. In one embodiment, the stent delivery system is similar to that shown in U.S. Pat. No. 6,432,133, which is incorporated by reference herein. A variety of imaging techniques (such as, but not limited to, high definition x-ray angiography, MRI angiography, and CT tomography) can be used, alone or in combination, to guide the stent delivery system. The markers are then used to place the portal substantially at the location of the proximal end of the branch artery while the stent is placed in the main artery.

In utilizing the stent of this teachings, the stent is guided along the main artery, utilizing an imaging technique and the one or more pairs of markers (indicating elements), the guiding enabling the placement of the stent and the locating of the portal (orifice) in the placement of the stent. Using the markers, the portal is placed substantially at the location of the proximal end of the branch artery.

In one instance, the method for delivering a stent of these teachings to a desired intraluminal location includes mounting the expandable stent on an expandable member, such as a balloon, provided on the distal end of an intravascular catheter, introducing the delivery catheter with the stent mounted on the inflatable balloon within a patience vasculature, advancing the catheter over a guide wire to the desired (predetermined) location within the patient's body lumen, inflating the balloon on the catheter to expand the stent into a permanent expanded condition and then deflating the balloon and removing the catheter,

In one embodiment, the stent of these teachings includes a substantially cylindrical expandable structure having two ends, a locus of points at one of the two ends defining a surface, the surface being beveled with respect to a central axis of the substantially cylindrical expandable structure.

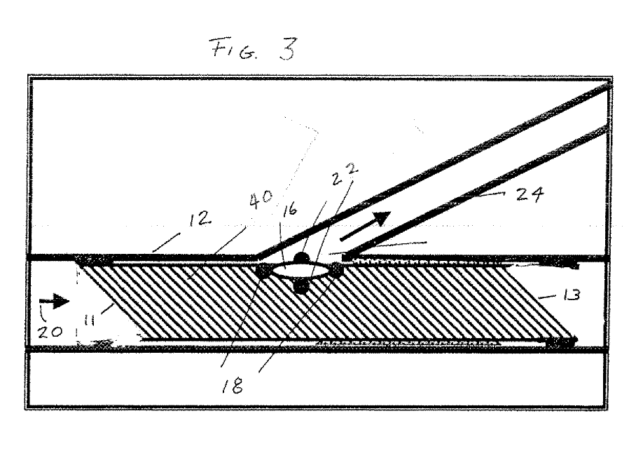

FIG. 3 shows an embodiment of the stent 40 of these teachings holding open the artery 12 after the catheter is withdrawn. Referring to FIG. 3, the stent 40 is a substantially cylindrical expandable structure having two ends 11, 13. A locus of points at one of the two ends 11, 13 defines a surface, which is beveled with respect to a central axis 20 of the stent 40. In the embodiment shown in FIG. 3, both ends 11, 13 define surfaces that are beveled. In the instance in which both surfaces are beveled, embodiments in which an angle between a normal to the first beveled surface and the central axis is different from an angle between a normal to the other beveled surface and the central axis are within the scope of these teachings.

In some instances, in conventional stent designs, the insertion of a catheter into the lumen of a stented vessel is difficult. The perpendicular end of a stent creates an abrupt transition between the vessel and the stent. A catheter inserted into the artery will encounter the pointed tips of the conventional stent lattices at the same position along the length of the artery. A common practice for cannulation is to use an angled catheter that can be rotated to move the distal end of the catheter away from obstructions, including the tips of the lattices located at the proximal end of the stent. However, the perpendicular cut of conventional stent increases the likelihood that the distal end of the catheter will collide with the proximal end of one or more lattices—even when the catheter is rotated. That is, the perpendicular cut of conventional stents creates an obstacle that challenges the current practices of cannulation. Great difficulty may be encountered when attempting to pass a catheter through the site because the catheter may be unable to turn within the tight radius, or the end of the catheter may become caught on the proximal edge of the conventional stent, or the end of the catheter may become caught between the outer surface of the conventional stent and the inner surface of the artery.

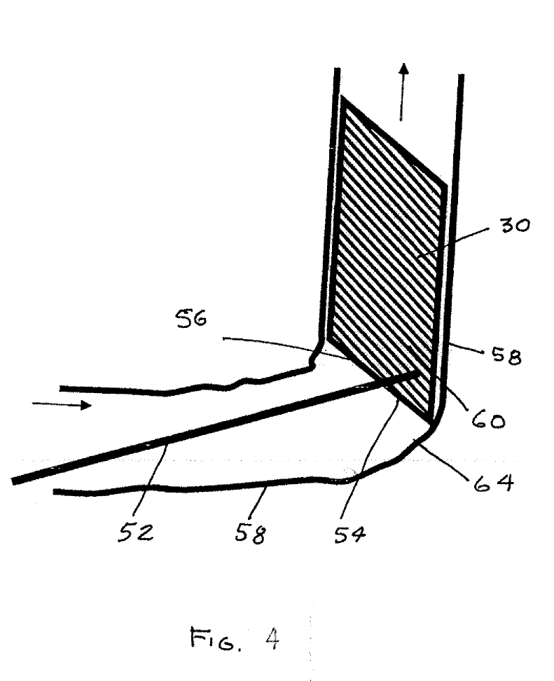

As shown in FIG. 4, an embodiment of the stent 40 of these teachings allows an angled catheter 52 to be placed into the proximal end 54 of the stent 40 with less difficulty than stents that feature perpendicular ends. The diagonal cut of the beveled end 56 of stent of these teachings 40 provides a more gradual transition between the vessel 58 and the stent 40. This beveled end 56 allows a catheter 52 to be inserted into the lumen 60 of the stent 30 and artery 58 without colliding with the proximal end 54 of the stent 40 or becoming entrapped between the outer surface of the stent 40 and the inner surface 64 of the artery 58.

In one embodiment, the stent of these teachings is covered with a synthetic material or when having structural elements, comprising synthetic material, interdigitated therein. In one embodiment, the synthetic material is selected from the group consisting of polyester, polytetra-fluoroethylene, microporous urethane, nylon, and lycra. The stent graft of these teachings can have structural elements interdigitated therein as described in U.S. patent application Ser. No. 10/423,370, also published as U.S. Patent Application Publication No. U.S. 2004/0215320 A1, both of which are incorporated by reference herein.

Other variations of the described teachings will occur to those skilled in the art given the benefit of the described teachings. The following claims define the scope of the teachings.

What is claimed is:

1. A stent comprising:
a substantially cylindrical expandable structure comprising a plurality of interconnected elements;
an outer surface of said substantially cylindrical expandable structure comprising an orifice, said other fees being capable of allowing flow between an internal volume of said substantially cylindrical expandable structure and an external volume.

2. The stent of claim 1 wherein a characteristic dimension of said orifice is less than a diameter of a branch vessel, wherein a proximal end of the branch vessel is located substantially at a location of said orifice.

3. The stent of claim 1 wherein a characteristic dimension of said orifice is selected to enable cannulation of a branch vessel, wherein a proximal end of the branch vessel is located substantially at a location of said orifice.

4. The stent of claim 1 wherein said outer surface comprises at least one pair of indicating elements.

5. The stent of claim 4 wherein said at least one pair of indicating elements comprises two pairs of indicating elements; one pair of said two pairs of indicating elements being disposed in a direction substantially parallel to a central axis of said substantially cylindrical expandable structure and also disposed on the opposite side of said orifice; another pair of said two pairs of indicating elements being disposed in a direction substantially perpendicular to a central axis of said substantially cylindrical expandable structure and also disposed on opposite sides of said orifice.

6. The stent of claim 5 wherein each indicating elements of said two pairs of indicating elements comprises a material capable of being detected by a predetermined imaging method, said predetermined imaging method being utilized for guiding placement of the stent.

7. The stent of claim 6 wherein said predetermined imaging method is at least one method selected from high definition x-ray angiography, MRI angiography, and CT tomography.

8. The stent of claim 6 wherein said material comprises tantalum.

9. The stent of claim 1 wherein said orifice is a substantially elliptical orifice.

10. The stent of claim 1 wherein said substantially cylindrical expandable structure comprises a shape memory alloy.

11. A method for placing a stent in a bifurcated vessel, the method comprising the steps of:
providing a stent comprising a substantially cylindrical expandable structure, an outer surface of the substantially cylindrical expandable structure comprising an orifice and at least one pair of indicating elements located on opposite sides of the orifice;
guiding, utilizing an imaging technique and the at least one pair of indicating elements, the placement of the stent and the location of the orifice in the placement of the stent;
delivering the stent to a predetermined intraluminal location; and
placing the orifice substantially at a proximal end of the branch vessel.

12. The stent of claim 1 wherein said substantially cylindrical expandable structure has two ends; a locus of points at one of said two ends defining a surface, said surface being beveled with respect to a central axis of said substantially cylindrical expandable structure.

13. The stent of claim 12 wherein a locus of points at another one of said two ends defines another surface, said another surface being beveled with respect to the central axis of said substantially cylindrical expandable structure.

14. The stent of claim 13 wherein an angle between a normal to said surface and said central axis is different from an angle between a normal to said another surface and said central axis.

15. The stent of claim 1 wherein said substantially cylindrical expandable structure is covered with a synthetic material.
